(19) United States
(12) Patent Application Publication
(10) Pub. No.: US 2026/0315121 A1
(43) Pub. Date: Oct. 15, 2026

(54) ORTHOPAEDIC DISTRACTION DEVICE FOR DISTRACTION OF A PATIENT'S JOINT, ADVANTAGEOUSLY THE KNEE

(71) Applicant: NEWCLIP INTERNATIONAL, LUXEMBOURG (LU)

(72) Inventors: Hugo PEIGNE, SAINT-SEBASTIEN-SUR-LOIRE (FR); Grégoire LARCHE, NANTES (FR); Jean-Pierre PODGORSKI, CLISSON (FR)

(21) Appl. No.: 19/481,265

(22) PCT Filed: May 2, 2024
(86) PCT No.: PCT/EP2024/062023

§ 371 Date: Nov. 4, 2025

(30) Foreign Application Priority Data
May 5, 2023 (FR) FR2304509

(51) Int. Cl. A61B 17/80

(52) U.S. Cl. CPC A61B 17/8004; A61B 17/8061

(57) ABSTRACT

The present invention relates to an orthopaedic distraction device for distraction of a patient's joint (A), which comprises:—securing means (2), for securing said orthopaedic distraction device (1) to the bones (S) that make up said joint (A),—telescopic connecting means (3), connecting said securing means (2) and defining a degree of freedom in translational movement (T),—manoeuvring means (4), suitable for generating a manoeuvre of spacing said securing means (2) apart, and—locking means (5), suitable for locking said telescopic connecting means (3) in terms of translation after said distraction has been created.

### TECHNICAL FIELD OF THE INVENTION

The present invention relates to the technical field of orthopaedic devices.

More particularly, it relates to orthopaedic distraction devices suitable for distraction of a patient's joint, advantageously the knee.

### STATE OF THE ART

Osteoarthritis of a joint, particularly the knee, is a degenerative joint disease characterised by persistent pain that can lead to loss of function.

Treatment options are currently limited. And standard treatments can only slow down the disease.

In this context, this disease generally leads to a prosthetic joint replacement surgery (e.g., in case of bicompartmental joint degeneration).

There is therefore a certain interest in surgical treatments capable of saving joints, particularly in young patients with osteoarthritis of the knee.

Within this framework, joint distraction could be an interesting solution in that it improves joint homeostasis by temporarily relieving mechanical stress on the joint.

But the current orthopaedic distraction devices have a number of disadvantages in achieving this distraction, including large space requirement, significant discomfort for the patient, external arrangement (leading to a high infection rate due to percutaneous pins) and complexity of fitting for the orthopaedic surgeon.

### DISCLOSURE OF THE INVENTION

In order to remedy the above-mentioned drawbacks of the state of the art, the present invention proposes an orthopaedic distraction device for distraction of a patient's joint, advantageously the knee.

The orthopaedic distraction device according to the invention, advantageously internal or external, comprises:
securing means, for securing said orthopaedic distraction device to the bones forming said joint,
said securing means comprising first securing means for the securing to at least one first bone of said joint, advantageously a tibia, and second securing means for the securing to at least one second bone of said joint, advantageously a femur,
   telescopic connecting means, connecting said securing means and defining a translational degree of freedom between said securing means,
   manoeuvring means, suitable for generating a manoeuvre of spacing said securing means apart according to said translational degree of freedom to create a distraction between the bones of said joint, and
   locking means, suitable for locking in translation said telescopic connecting means after said distraction has been created.

Such an orthopaedic distraction device according to the invention shows various advantages, i.e. in particular reduced space requirement, improved comfort for the patient, versatile arrangement possibility (possibly external and preferably internal, which eliminates the risk of infection associated with percutaneous pins) and easy fitting for the orthopaedic surgeon.

Other non-limiting and advantageous features of the product according to the invention, taken individually or according to all the technically possible combinations, are the following:
the telescopic connecting means comprise at least two arms that form together a slide defining said translational degree of freedom, said arms each having a proximal end that is connected to one of said securing means; preferably, said at least two arms comprise a first arm, having a tubular wall that defines an elongated housing opening into an aperture, and a second arm, fitted into said elongated housing through said aperture and guided in translation along said elongated housing; also preferably, the locking means comprise at least one stop screw that is guided in translation on one of said arms and that cooperates with the other of said arms to ensure said locking, advantageously among an axial stop screw, to maintain the distraction, preferably implanted parallel to the translational degree of freedom, advantageously in a bottom of the elongated housing located opposite the aperture, and a side stop screw, to lock the translational clearance, preferably implanted perpendicular to the translational degree of freedom, advantageously into the tubular wall of the first arm; the manoeuvring means advantageously comprise a cam, that is removable and free to rotate, to generate a relative displacement of said arms by a rotational movement; preferably, the removable cam is assembled on a second arm and cooperates with a first arm; also preferably, the removable cam is assembled on the second arm, preferably at its proximal end, and cooperates with the tubular wall of the first arm, on the side of its aperture; the manoeuvring means advantageously comprise stop means, to define the end-of-rotational travel position of said cam, e.g. a couple of lugs, including a first lug carried by said cam and a second lug carried by the tubular wall of the first arm; as an alternative and/or a complement, the manoeuvring means comprise an axial manoeuvring screw that is guided in translation on one of said arms, parallel to the translational degree of freedom (advantageously in a bottom of the elongated housing located opposite the aperture), and that cooperates with the other of said arms to ensure the spacing manoeuvre;
the locking means comprise complementary securing means forming a spacer, which are shaped to bridge the joint and to be attached to an opposite bone of said joint;
the securing means consist of two osteosynthesis plates, i.e. a first osteosynthesis plate, forming the first securing means intended to be secured to the first bone of said joint, and a second osteosynthesis plate, forming the second securing means intended to be secured to the second bone of said joint, said osteosynthesis plates advantageously including holes suitable for fitting fixing screws; preferably, the proximal end of an arm is integral with the associated osteosynthesis plate, or assembled with the osteosynthesis plate, for example by screwing;
the orthopaedic distraction device comprises angulation means for providing a rotational degree of freedom between said securing means, perpendicular to said translational degree of freedom, in order to allow a distraction with the joint in flexed state; the angulation means advantageously comprise articulation means that are placed between one of the securing means and the telescopic connecting means, and rotational locking means, for locking said articulation means in rotation after said angulation has been adjusted, e.g. in the form of complementary toothed gears associated with a clamping member.

The present invention also relates to the orthopaedic distraction method, implementing an orthopaedic distraction device according to the invention, said method comprising:
a preliminary surgical operation applied to at least one of the bones forming said joint,
a step of positioning and securing said orthopaedic distraction device to the bones forming said joint,
possibly a step of generating an angulation between the securing means,
a step of creating the distraction by implementing manoeuvring means that cause a manoeuvre spacing the securing means apart,
a step of maintaining the distraction, by implementing the locking means.

Obviously, the different features, alternatives and embodiments of the invention can be associated with each other according to various combinations, insofar as they are not incompatible or exclusive with respect to each other.

### DETAILED DESCRIPTION OF THE INVENTION

Moreover, various other features of the invention emerge from the appended description made with reference to the drawings that illustrate non-limiting embodiments of the invention, and wherein:

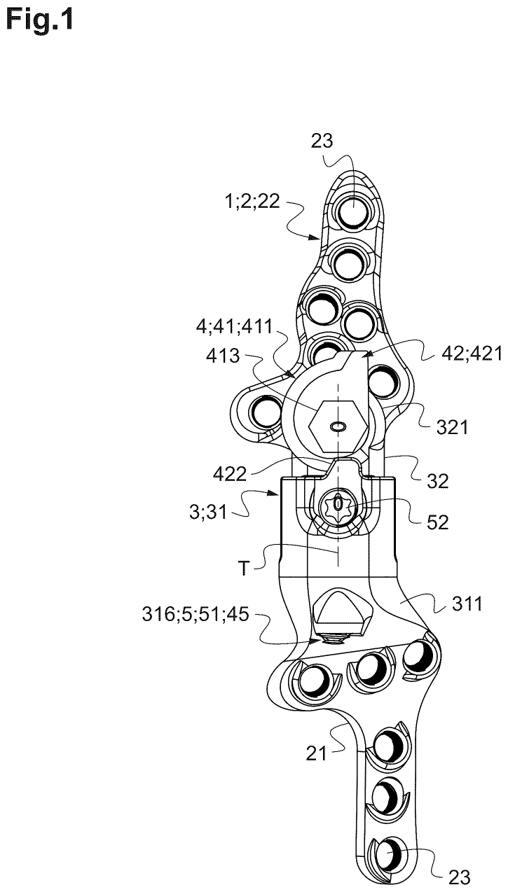

FIG. 1 is an overall view of a first orthopaedic distraction device according to the invention;

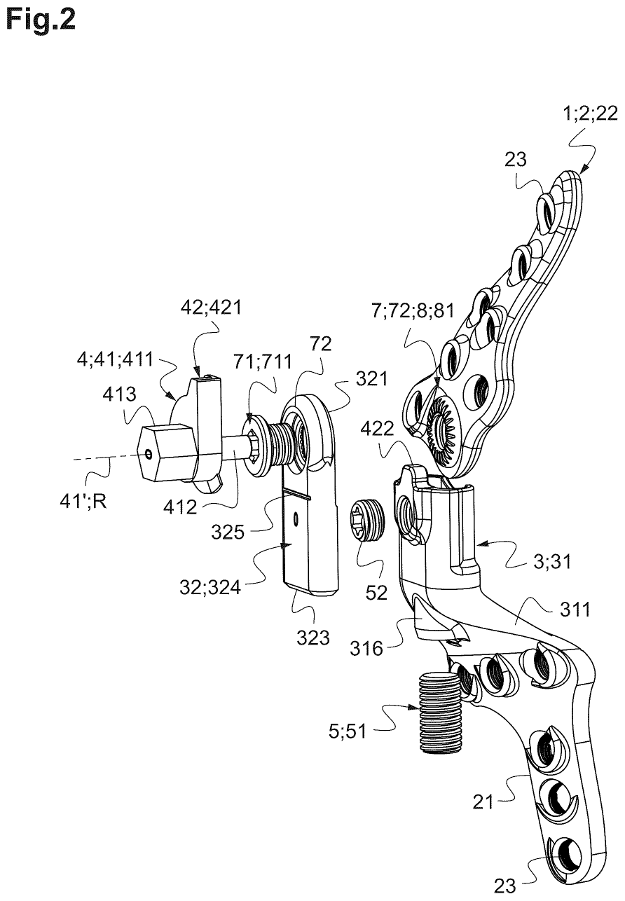

FIG. 2 is an exploded overall view of the first orthopaedic distraction device according to FIG. 1;

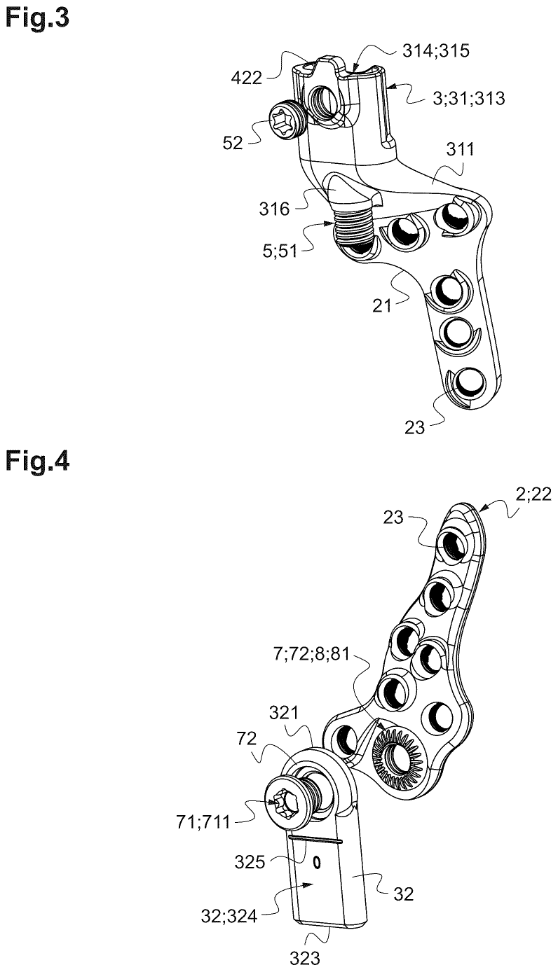

FIG. 3 is an overall view of a first part of the orthopaedic distraction device according to FIG. 1, i.e. a first arm (including an elongated housing) with its osteosynthesis plate here intended to be secured to the tibia;

FIG. 4 is an overall view of a second part of the orthopaedic distraction device according to FIG. 1, i.e. a second arm (intended to be fitted into an elongated housing) with its osteosynthesis plate here intended to be secured to the femur;

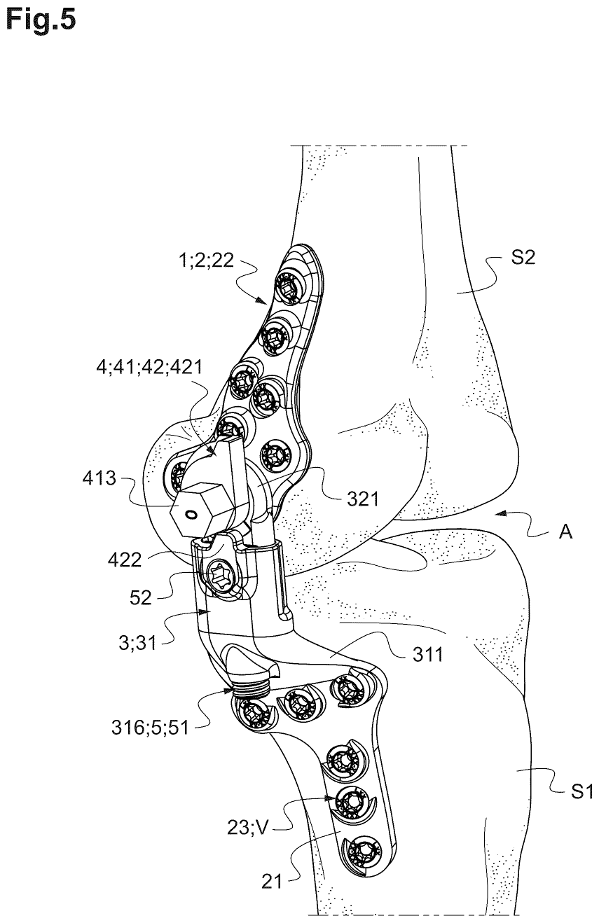

FIG. 5 is a perspective overall view of the orthopaedic distraction device according to FIG. 1, secured to the bones forming the joint and previously to the distraction creation step;

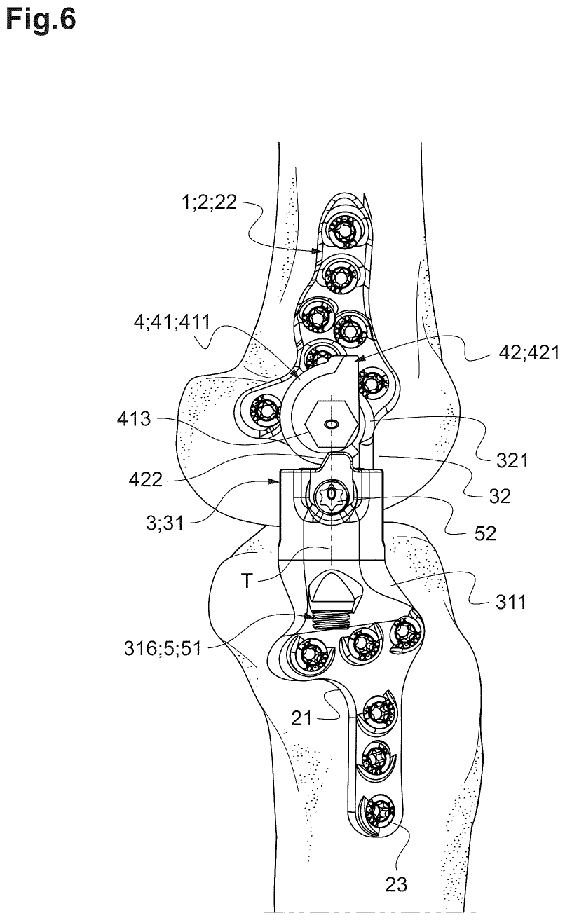

FIG. 6 is a front overall view of the orthopaedic distraction device according to FIG. 5;

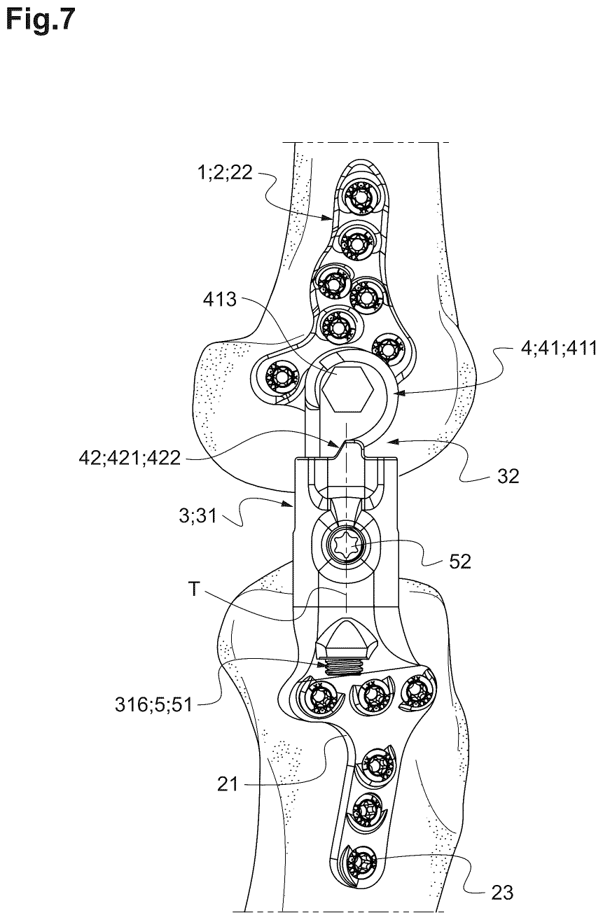

FIG. 7 is an overall view of the orthopaedic distraction device according to FIG. 1, secured to the bones forming the joint and following the distraction creation step;

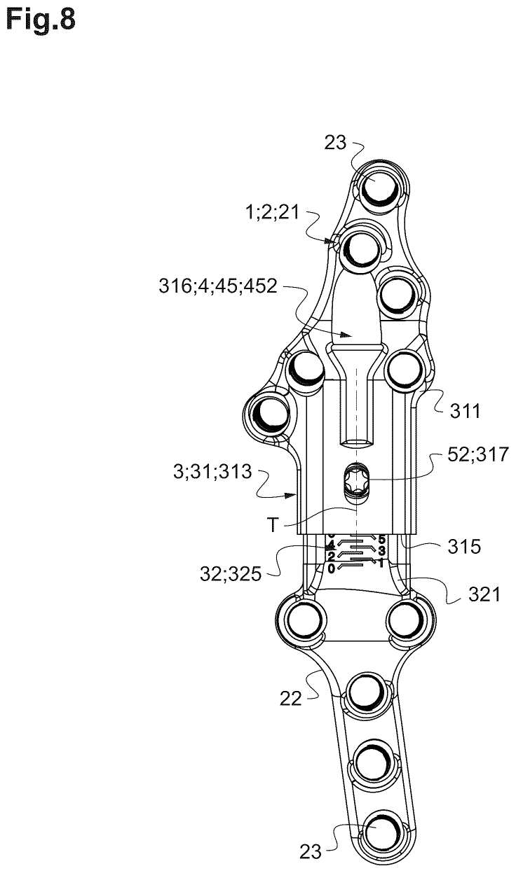

FIG. 8 is an overall view of a second orthopaedic distraction device according to the invention;

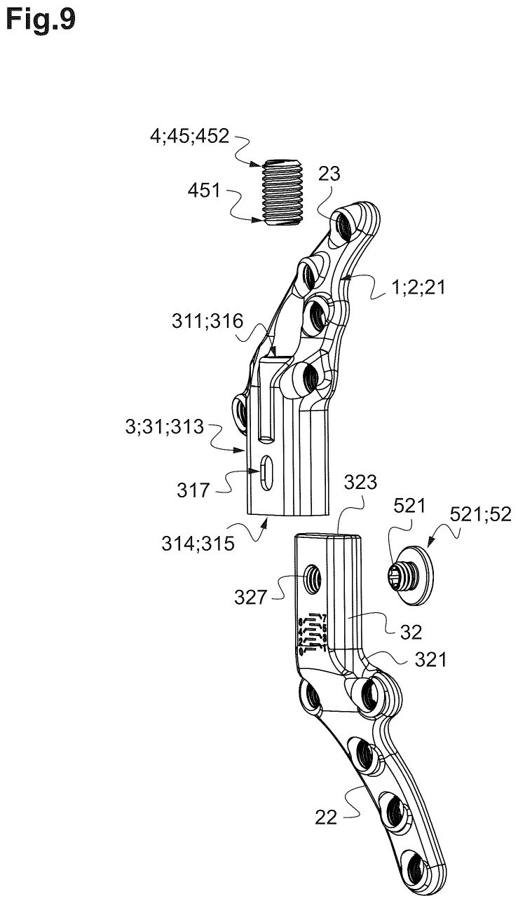

FIG. 9 is an exploded overall view of the second orthopaedic distraction device according to FIG. 8;

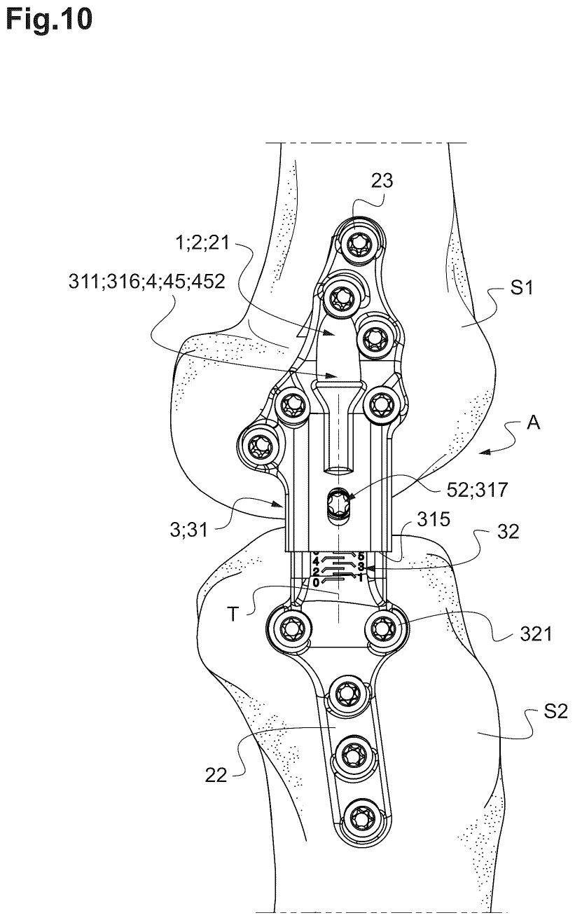

FIG. 10 is an overall view of the orthopaedic distraction device according to FIG. 8, secured to the bones forming the joint;

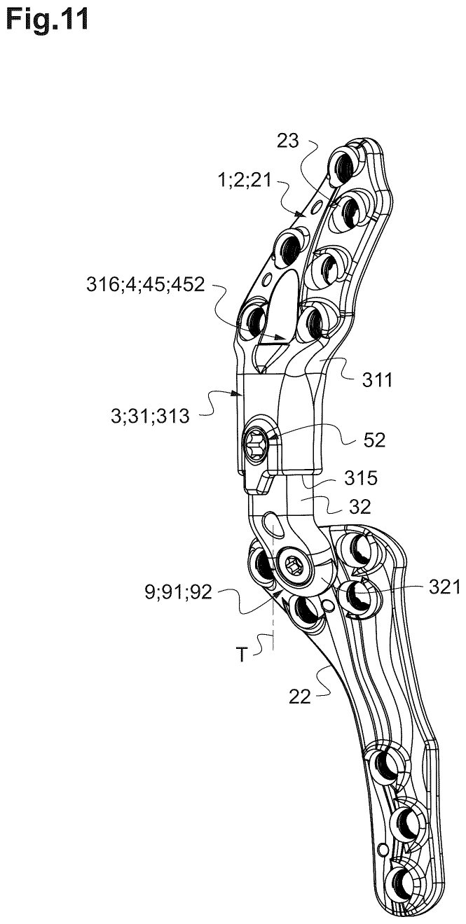

FIG. 11 is an overall view of a third orthopaedic distraction device according to the invention;

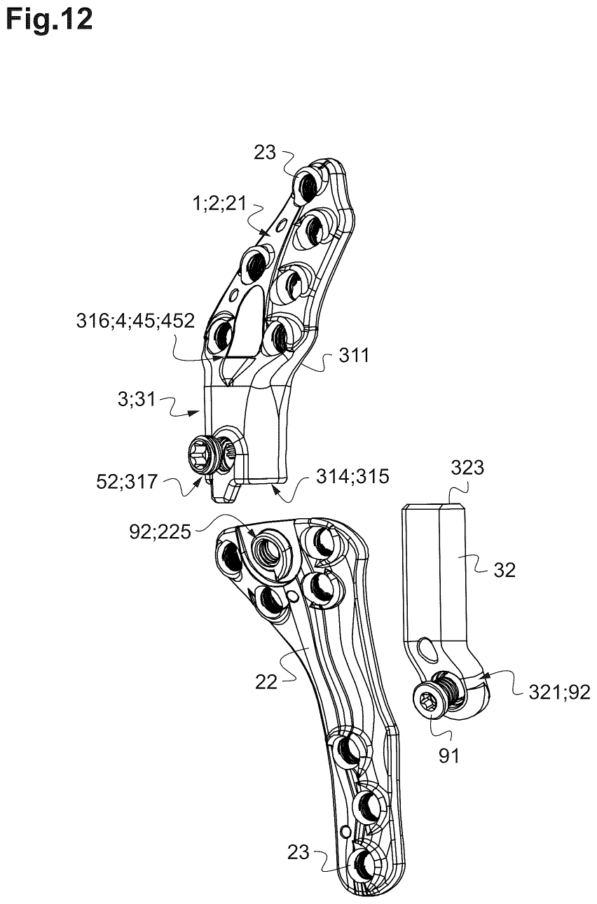

FIG. 12 is an exploded overall view of the third orthopaedic distraction device according to FIG. 11;

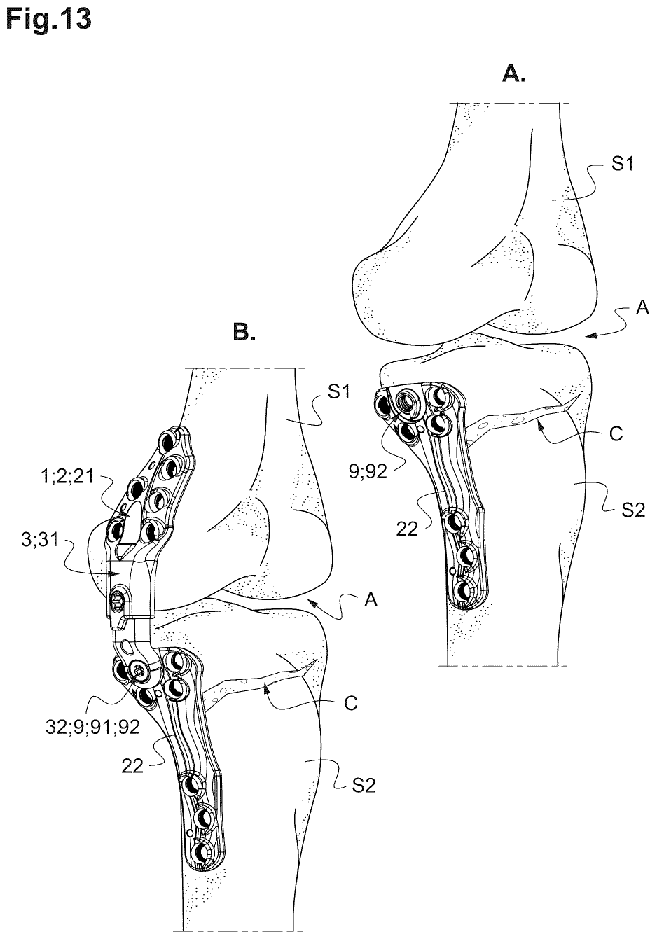

FIG. 13 is an overall view of the orthopaedic distraction device according to FIGS. 11 and 12, secured to the bones forming the joint, showing the implantation before distraction (part A) and after distraction (part B);

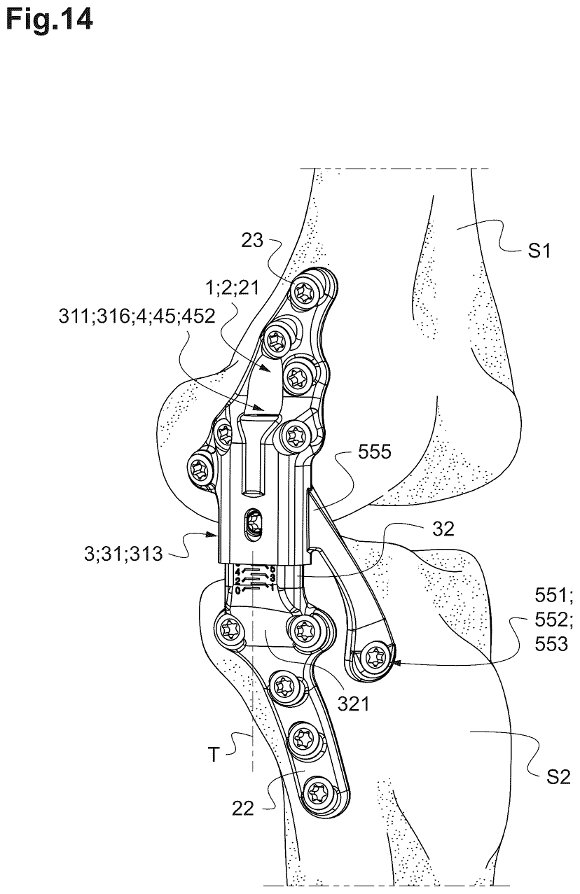

FIG. 14 is an overall view of a fourth orthopaedic distraction device according to the invention, which differs from the second orthopaedic distraction device in that it has complementary securing means forming spacer.

It is to be noted that, in these figures, the structural and/or functional elements common to the different alternatives can have the same references numbers.

As described hereinafter in relation with the figures, the present invention thus relates to an orthopaedic distraction device 1 designed for the distraction of a patient's joint A, advantageously the knee.

By “distraction”, it is meant a temporary mechanical relief of the joint A by creating a spacing between the two bones S forming this joint A.

For example, as illustrated in FIGS. 5 to 7 or FIG. 10 in the case of a knee, the temporary distraction is implemented between the tibia S1 and the femur S2, advantageously with a spacing of the order of 5 mm and maintained between 6 and 8 weeks.

Herein, the orthopaedic distraction device 1 advantageously consists of a surgical or implantable orthopaedic distraction device 1.

The orthopaedic distraction device 1 is here internal, advantageously implanted under the patient's skin and soft tissues, which eliminates the risk of infection associated with percutaneous pins.

As an alternative, the orthopaedic distraction device 1 is external, advantageously positioned outside the patient's skin, with securing means intended to be implanted through the skin and the soft tissues.

Generally, according to the invention, the orthopaedic distraction device 1 comprises:
securing means 2, for securing the orthopaedic distraction device 1 to the bones S forming the joint A,
telescopic connecting means 3, connecting the securing means 2 and defining a translational degree of freedom T between the securing means 2,
manoeuvring means 4, suitable for generating a manoeuvre of spacing the securing means 2 apart according to the translational degree of freedom T to create the distraction between the bones S of the joint A, and
locking means 5, suitable for locking the telescopic connecting means 3 in translation after the desired distraction has been created.

In particular, the translational degree of freedom T advantageously extends perpendicular to the bending axis of the joint A or to the joint space.

### Securing Means

The securing means 2 comprise:
first securing means 21 for the securing to at least one first bone S1 of the joint A (advantageously a tibia for the knee), and
second securing means 22 for the securing to at least one second bone S2 of the joint A (advantageously a femur for the knee).

Preferably, the securing means 2 consist of two osteosynthesis plates 2, i.e.:
a first osteosynthesis plate 21, forming the first securing means 21 intended to be secured to at least one first bone S1 of the joint A, and
a second osteosynthesis plate 22, forming the second securing means 22 intended to be secured to at least one second bone S2 of the joint A.

Generally, for the knee, the first securing means 21 are suitable for being secured to the tibia, forming tibial securing mean 21; and the second securing means 22 are suitable for being secured to the femur, forming femoral securing means 22.

As an alternative, as illustrated in FIG. 8 and following, the securing means 2 are suitable for an inverted assembly compared to FIG. 5 and following:
the first securing means 21 are suitable for a securing to a first bone S1, which is here the femur, forming femoral securing means 21, and
the second securing means 22 are suitable for a securing to a second bone S1, which is here the tibia, forming tibial securing means 22.

Generally, the osteosynthesis plates 2 advantageously includes holes 23 suitable for fitting fixing screws V into the bone S.

Preferably, the holes 23 are arranged so that the fixing screws V protect the joint surfaces and so that the fixing screws V diverge to increase the anchoring force.

Conventionally, such osteosynthesis plates 2 include:
a rear face, advantageously anatomical and intended to come into contact with the bone, and
an opposite, front face.

Such an osteosynthesis plate 2 is advantageously a few millimetres thick.

Still generally, as illustrated in particular in FIGS. 5 to 7, the securing means 2 are advantageously shaped for attachment to the medial and/or lateral and/or anteromedial and/or anterior surfaces of the bones S of the joint A.

### Telescopic Connecting Means

The telescopic connecting means 3 advantageously comprise at least two arms 31, 32 that form together a slide defining the translational degree of freedom T.

The arms 31, 32 each have a proximal end 311, 321 that is linked (or connected) to one of the securing means 2.

Herein, a first arm 31 cooperates with the first securing means 21 (FIGS. 2 and 3, or also FIG. 8 and following).

The first arm 31 (advantageously in the form of a sleeve or bushing) advantageously includes a tubular wall 313 that defines an elongated housing 314, advantageously a blind housing, opening into an aperture 315.

The elongated housing 314 advantageously consists of a blind housing, having a bottom 316 located opposite the aperture 315.

The elongated housing 314 advantageously extends coaxially relative to the translational degree of freedom T.

A second arm 32, cooperating with the second securing means 22, can be seen in more detail in FIGS. 2 and 4 or also in FIG. 8 and following.

The second arm 32 is intended to be fitted into the elongated housing 314 of the first arm 31, through the aperture 315 thereof and guided in translation along the elongated housing 314 (coaxially to the translational degree of freedom T).

In particular, the cross-section of the second arm 32 corresponds, to within the clearance, to the cross-section of the elongated housing 314 of the first arm 31 so as to ensure the relative translational guiding between these two arms 31, 32 according to the translational degree of freedom T.

Moreover, the second arm 32 advantageously includes a visual marker 325 which is useful for observing/measuring the movement of this second arm 32 with respect to the aperture 315 of the first arm 31, corresponding to the distraction applied to the joint A.

Generally, for the knee, the first arm 31 cooperates for example with first “tibial” securing means 21 and the second arm 32 cooperates with second “femoral” securing means 22 (e.g., FIGS. 1 to 7).

As an alternative, in the case of an inverted assembly compared to that shown in FIG. 5 and following (FIG. 8 and following), the first arm 31 cooperates with first “femoral” securing means 21 and the second arm 32 cooperates with second “tibial” securing means 22.

Generally, the telescopic connecting means 3 can be made integral with the securing means 2 (FIGS. 9 to 11) or assembled thereto (FIGS. 1 to 8 or FIGS. 11 and 12).

In practice, the proximal end 311, 321 of an arm 31, 32 is advantageously:
integral with the associated osteosynthesis plate 21, 22 (FIGS. 9 to 11), or
assembled to the associated osteosynthesis plate 21, 22, for example by screwing (FIGS. 1 to 8 or FIGS. 11 and 12).

For example, according to FIGS. 1 to 7, or 11 and 12, the proximal end 311 of the first arm 31 is integral with the first osteosynthesis plate 21. The proximal end 321 of the second arm 32 is assembled to the associated second osteosynthesis plate 22.

Generally, the assembly means 7, 9 (between the proximal end 321 of the second arm 32 and the associated second osteosynthesis plate 22) are advantageously chosen among:
articulation means 7 being part of angulation means 6 described hereinafter in relation to FIGS. 1 to 7, providing a rotational degree of freedom between the proximal end 321 of the second arm 32 and the associated of the second osteosynthesis plate 22, or
fixed assembly means 9, described hereinafter in relation to FIGS. 11 to 13, defining an accurate angular position between the second arm 32 and the associated second osteosynthesis plate 22.

For the “fixed assembly means 9” embodiment, the second osteosynthesis plate 22 advantageously includes a location 225 intended to receive the proximal end 321 of the second arm 32 and to ensure the angular locking of this second arm 32.

Generally, to carry out the assembly, the proximal end 321 of the second arm 32 is advantageously:
superimposed to a front face of the second osteosynthesis plate 22, then
assembled using a clamping member 71, 91, e.g. a screw, that is fitted through a couple of aligned holes 72, 92 that are arranged respectively in the arm 32 (at the proximal end 321 thereof) and in the associated osteosynthesis plate 22.

According to the embodiment illustrated in FIGS. 1 to 7 or FIGS. 11 and 12, the second osteosynthesis plate 22 preferably consists of an osteosynthesis plate suitable to be fitted on a bone S1 subsequent to a surgical procedure applied to said bone S2 (e.g. osteosynthesis of a fracture or corrective osteotomy, as illustrated in FIG. 13).

As an alternative, according to FIGS. 8 to 10, the proximal end 311 of the first arm 31 is integral with the first osteosynthesis plate 21. The proximal end 321 of the second arm 32 is integral with the associated second osteosynthesis plate 22.

### Manoeuvring Means

According to a first embodiment illustrated in FIGS. 1 to 7, the manoeuvring means 4 advantageously consist of a cam 41, here removable and free to rotate, to generate a relative displacement of said arms 31, 32 by its rotational movement.

Preferably, the removable cam 41 is temporarily assembled to the second arm 32, preferably at the proximal end 321 thereof.

And this cam 41 cooperates with the tubular wall 313 of the first arm 31, on the side of aperture 315 thereof.

Such a removable cam 41 is interesting in particular to limit the space required for the orthopaedic distraction device 1, in particular when it is implanted internally.

This “removable” structure also makes it possible to chose the cam 41 among a range of cams 41 that have respective distraction values and that make it possible to take into account a potential variation of initial length at the telescopic connecting means 3.

Herein, the cam 41 advantageously consists of a cam with an external profile whose rotation about its axis of rotation 41′ generates a radial effort (perpendicular to the axis of rotation 41′) between the arms 31, 32.

For that purpose, the cam 41 has an external profile 411 that is curved and that gradually moves away from the axis of rotation 41′.

For its guiding in rotation, the cam 41 advantageously includes a cylindrical section 412 that is intended to cooperate with the second arm 32 to define the rotational degree of freedom.

Herein, the cylindrical section 412 is intended to come inside a housing located at the proximal end 321 of the second arm 32.

This housing is here formed by the female head 711 of the above-mentioned clamping member 71.

For its rotational manoeuvring, the cam 41 advantageously includes a gripping member 413, here centred on the rotation axis 41′, suitable for cooperating with a manoeuvring tool.

This gripping member 413 is here in the form of a hexagonal head.

Also preferably, the manoeuvring means 4 comprise stop means 42, to define the end-of-rotational travel position of the cam 41 during distraction.

For example, the stop means 42 comprise a couple of lugs 421, 422 intended to cooperate in the end-of-rotational travel position of the cam 41.

Herein, the couple of lugs 421, 422 comprises:
a first lug 421 carried by the cam 41, at the end of its external profile 411, and
a second lug 422, here in protrusion, carried by the tubular wall 313 of the first arm 31 (on the side of aperture 315 thereof).

The second lug 422 here sandwiches the cam 41 (at the external profile 411 thereof) with the second arm 32, so as to help holding the cam 41 when the latter is rotated. In other words, the second lug 422 defines a groove with the second arm 32, which is intended to receive and guide the external profile 411, to help holding the cam 41.

The second lug 422 can possibly serve as a landmark with respect to the visual marking 325, so as to check the distraction size applied to the joint A.

According to a second embodiment illustrated in particular in FIG. 8 and following, the manoeuvring means 4 comprise an axial manoeuvring screw 45 that is guided in translation on one of the arms 31, parallel to the translational degree of freedom T.

Such an axial manoeuvring screw 45 has for interest to eliminate additional instruments compared to a cam 41, thus simplifying the operating technique.

Preferably, this axial manoeuvring screw 45 is implanted parallel to the translational degree of freedom T, advantageously in the bottom 316 of the elongated housing 314 of the first arm 31.

Preferably, this axial manoeuvring screw 45 is intended to travel in translation along the translational degree of freedom T.

This axial manoeuvring screw 45 advantageously comprises two ends (FIG. 9):
a first end 451, located within of the elongated housing 314 and intended to be pressed on an end wall 323 of the second arm 32 for the translational manoeuvre thereof, and
a second end 452, located outside the elongated housing 314 and forming a rotational manoeuvring head.

Generally, the rotational manoeuvre of the axial manoeuvring screw 45 (screwing or unscrewing) generates a manoeuvre of the arms 21, 22 relative to each other, leading to a manoeuvre of spacing the securing means 2 apart along the translational degree of freedom T to create a distraction between the bones S of the joint A.

### Locking Means

The locking means 5 advantageously comprise at least one stop screw 51, 52 that is guided in translation on one of the arms 31, 32 and that cooperates with the other of the arms 31, 32 to provide this locking.

For example, the locking means 5 comprise at least one stop screw 51, 52 among:
an axial stop screw 51, to maintain the applied distraction, and
a side stop screw 52, to lock the translational clearance.

The axial stop screw 51 provides an end-of-travel stop for the first arm 31 relative to the second arm 32.

Preferably, this stop screw 51 is implanted parallel to the translational degree of freedom T, advantageously in the bottom 316 of the elongated housing 314 of the first arm 31 to serve as an bearing stop for an end wall 323 of the second arm 32.

Generally, according to the first embodiment illustrated in FIGS. 1 to 7, the stop screw 51 is advantageously identical to the axial manoeuvring screw 45 according to FIG. 8 and following, to form manoeuvring means 4 suitable for being manoeuvred in complement (or combination) with the cam 41 in order to adjust the distraction.

The side stop screw 52 is preferably implanted perpendicular to the translational degree of freedom T.

This side stop screw 52 is for example implanted:
in the tubular wall 313 of the first arm 31, so as to come and exert a compression on a side wall 324 of the second arm 32 (FIGS. 1 to 7), or
in the second arm 32, within a tapped through-hole 327, so as to exert a friction pressure on the first arm 31 (FIGS. 8 to 10).

For a side stop screw 52, carried by the second arm 32, said side stop screw 52 advantageously includes:
a bearing plate 521, intended to exert the friction force on the tubular wall 313 of the first arm 31, and
a manoeuvring head 522, advantageously accessible through a window 317 arranged in the tubular wall 313 of the first arm 31.

As an alternative or a complement, as illustrated in FIG. 14, the locking means 5 advantageously comprise complementary securing means 55 forming a spacer, which are shaped to bridge the joint A and to be attached to an opposite bone S1, S2 of this joint A.

In other words, the complementary securing means 55 advantageously consist of a spacer that has a fixed length and that is secured to the two bones S1, S2 forming the joint A.

As developed hereinafter, this complementary attachment is implemented after the distraction step (after the translation).

Such complementary securing means 55 can be implemented in various embodiments according to the invention.

In other words, the complementary securing means 55 advantageously comprise complementary securing means 55 fitting:
the first securing means 21 and/or the first arm 31 cooperating with a bone S, for the securing with another bone S of the joint A (FIG. 14), and/or
the second securing means 22 and/or the second arm 32 cooperating with a bone S, for the securing with another bone S of the joint A (not shown).

For example, as illustrated in FIG. 14, the complementary securing means 55 extend from the first arm 31 cooperating with a bone S2, in particular from its tubular wall 313. These complementary securing means 55 bridge the joint A and are attached to an opposite bone S1 of this joint A.

The complementary securing means 55 advantageously comprise at least one arm 551 (or a strut), designed to span or bridge the joint A.

This arm 551 advantageously includes at least one hole 552 for the fitting of a fixing screw 553 to be attached to the opposite bone segment S1.

This arm 551 advantageously has a fixed length.

This arm 551 preferably diverges relative to the translational degree of freedom T, from its upstream end 555 (here, integral with the first arm 31).

### Angulation Means

According to the invention, as illustrated in FIGS. 1 to 7, the orthopaedic distraction device 1 advantageously comprises angulation means 6 to provide a rotational degree of freedom between the securing means 2, about an axis of rotation R that extends perpendicular to the translational degree of freedom T.

This technical feature then allows, if need be, a distraction with the joint A in flexed state.

According to a preferred embodiment, illustrated in FIG. 2, the angulation means 6 comprise:
articulation means 7 that are arranged between one of the securing means 2 and the telescopic connecting means 3, to allow the relative angulation about the axis of rotation R, and
rotational locking means 8, suitable for locking in rotation the articulation means 7 after the angulation about the axis of rotation R has been adjusted.

Herein, the articulation means 7 define the above-mentioned axis of rotation R.

The articulation means 7 comprise for example a clamping member, 71, e.g. a screw, that is fitted through a couple of holes 72 that are arranged respectively in an arm 32 (at the proximal end 321 thereof) and in its associated osteosynthesis plate 22.

The rotational locking means 8 consist for example of a couple of complementary and front toothed gears 81, forming a serrated wheel, which cooperates with the clamping member 71.

These serrated gears 81 are arranged around the above-mentioned holes 72 and at the facing surfaces to engage them when the clamping member 71 is tightened.

In other words, the tightening of the clamping member 71 allows the toothed gears 81 to be engaged, which causes a locking in rotation between the arm 32 and its associated osteosynthesis plate 22.

### Orthopaedic Distraction Method

An orthopaedic distraction method, implementing the orthopaedic distraction device 1 according to the invention, will now be described in relation with FIGS. 5 to 7.

The orthopaedic distraction method 1 comprises:
a step of positioning and securing the orthopaedic distraction device 1 to the bones S forming the joint A (FIGS. 5 and 6),
possibly, and if need be, a step of generating an angulation between the securing means 2,
a step of creating the distraction, advantageously of the order of 5 mm, by implementing the manoeuvring means 4 that cause a manoeuvre of spacing the securing means 2 apart (from FIG. 6 to FIG. 7), then
a step of maintaining the distraction, by implementing the locking means 5.

The orthopaedic distraction device 1 according to the invention has for interest to here enable a step of internal positioning and securing, advantageously under the skin and soft tissues.

Herein, the orthopaedic distraction device 1 is positioned and locked on either side of the joint A by means of screws V fitted on the two osteosynthesis plates 2.

According to the embodiment illustrated in FIGS. 1 to 7, an angulation step is enabled by the angulation means 6, by acting on the rotational locking means 8 (here tightening/loosening the clamping member 71).

Again according to the first embodiment, the removable manoeuvring means 4 can here be temporarily fitted for implementing the distraction creation step, and then be removed after the distraction maintaining step.

As the case may be, the cam 41 is rotated, preferably up to the end-of-rotational travel position, to generate the desired distraction.

The distraction maintaining step is here carried out by successively manoeuvring the two stop screws 51:
an axial stop screw 51, to maintain the applied distraction, and
a side stop screw 52, to lock the translational clearance.

The cam 41 is in particular removed after the axial stop screw 51 has been tightened.

According to the second embodiment (FIG. 8 and following), the axial manoeuvring screw 45 is rotated, preferably screwed, to generate the desired distraction.

The distraction maintaining step is here carried out by a combined action of the axial manoeuvring screw 45 and the side stop screw 52, intervening in the locking of the translational clearance.

As the case may be, according to the fourth embodiment described in relation to FIG. 14, the distraction maintaining step is also carried out by implementation of the complementary securing means 55.

These complementary securing means 55 are intended to bridge the joint A by being attached to the opposite bone segment.

Once secured, these complementary securing means 55 act as a spacer (or a strut) between the two bones S1, S2 forming the joint A.

Moreover, the method can preferably comprise a preliminary surgical operation applied to one at least of the bones S1, S2 forming the joint A, for example osteosynthesis of a fracture or corrective osteotomy.

This preliminary surgical operation is in particular allowed by the orthopaedic distraction device 1 according to FIGS. 1 to 7, or FIGS. 11 and 12.

In this sense, a preliminary surgical operation C (e.g. osteosynthesis of a fracture or corrective osteotomy) is performed on the first bone S1 of the joint A (advantageously a tibia for the knee).

This preliminary surgical operation is stabilized by the implantation of the second securing means 22 on the first bone S1 (FIG. 13, part A).

The other components of the orthopaedic distraction device 1 are then fitted to carry out the step of positioning and securing the orthopaedic distraction device 1 to the bones S forming the joint A (FIG. 13, part B).

In particular, the proximal end 321 of the second arm 32 is secured to the second securing means 22 secured to the first bone S1 using the assembly means 7, 9. And the first securing means 21 carrying the first arm 31 are secured to the second bone S2.

The practitioner can then continue the method:
a step of creating the distraction, advantageously of the order of 5 mm, by implementing the manoeuvring means 4 that cause a manoeuvre of spacing the securing means 2 apart, then
a step of maintaining the distraction, by implementing the locking means 5.

Generally, at the end of the distraction period (generally a few weeks), the orthopaedic distraction device 1 is intended to be subject to a removal process.

Preferably, the removal process is carried out in the inverse direction compared to the orthopaedic distraction process 1:
a step of unlocking the distraction, by unlocking the locking means 5,
a step of detaching the orthopaedic distraction device 1 from the bones S forming the joint A.

Of course, various other modifications can be made to the invention within the scope of the appended claims.

1-13. (canceled)

14. An orthopaedic distraction device for distraction of a patient's joint,
wherein said orthopaedic distraction device comprises:
securing means, for securing said orthopaedic distraction device to the bones forming said joint,
said securing means comprising first securing means for the securing to at least one first bone of said joint, and second securing means for the securing to at least one second bone of said joint,
wherein said securing means consist of two osteosynthesis plates, i.e. a first osteosynthesis plate, forming the first securing means intended to be secured to the first bone of said joint, and a second osteosynthesis plate, forming the second securing means intended to be secured to the second bone of said joint,
telescopic connecting means, connecting said securing means and defining a translational degree of freedom between said securing means,
manoeuvring means, suitable for generating a manoeuvre spacing said securing means apart according to said translational degree of freedom to create a distraction between the bones of said joint, and
locking means, suitable for locking said telescopic connecting means in translation after said distraction has been created.

15. The orthopaedic distraction device according to claim 14, wherein the telescopic connecting means comprise at least two arms that form together a slide defining said translational degree of freedom,
wherein said arms each have a proximal end that is connected to one of said securing means.

16. The orthopaedic distraction device according to claim 15, wherein said at least two arms comprise:
a first arm, having a tubular wall that defines an elongated housing opening into an aperture, and
a second arm, fitted into said elongated housing through said aperture and guided in translation along said elongated housing.

17. The orthopaedic distraction device according to claim 15, wherein the manoeuvring means comprise a cam, that is removable and free to rotate, to generate a relative displacement of said arms by a rotational movement.

18. The orthopaedic distraction device according to claim 17, wherein said removable cam is assembled to a second arm and cooperates with a first arm.

19. The orthopaedic distraction device according to claim 15, wherein the manoeuvring means comprise an axial manoeuvring screw that is guided in translation on one of said arms, parallel to the translational degree of freedom, and that cooperates with the other of said arms to ensure the spacing manoeuvre.

20. The orthopaedic distraction device according to claim 19, wherein said at least two arms comprise:
a first arm, having a tubular wall that defines an elongated housing opening into an aperture, and
a second arm, fitted into said elongated housing through said aperture and guided in translation along said elongated housing,
wherein said axial manoeuvring screw is in a bottom of the elongated housing located opposite the aperture.

21. The orthopaedic distraction device according to claim 15, wherein the locking means comprise at least one stop screw that is guided in translation on one of said arms and that cooperates with the other of said arms to ensure said locking.

22. The orthopaedic distraction device according to claim 21, wherein said at least one stop screw is chosen among:
an axial stop screw, to maintain the distraction, and
a side stop screw, to lock the translational clearance.

23. The orthopaedic distraction device according to claim 15, wherein the proximal end of an arm is:
integral with the associated osteosynthesis plate, or
assembled to the osteosynthesis plate.

24. The orthopaedic distraction device according to claim 14, wherein the locking means comprise complementary securing means forming a spacer, which are shaped to bridge the joint and to be attached to an opposite bone of said joint.

25. The orthopaedic distraction device according to claim 14, wherein it comprises angulation means for providing a rotational degree of freedom between said securing means, perpendicular to said translational degree of freedom, in order to allow a distraction with the joint in flexed state.

26. The orthopaedic distraction device according to claim 25, wherein the angulation means comprise:
articulation means that are arranged between one of the securing means and the telescopic connecting means, and
rotational locking means, suitable for locking said articulation means in rotation after said angulation has been adjusted.

27. Orthopaedic distraction method for distraction of a patient's joint formed by bones, implementing an orthopaedic distraction device according to claim 14, said method comprising:
a preliminary surgical operation applied to at least one of the bones forming said joint,
a step of positioning and securing said orthopaedic distraction device to the bones forming said joint,
a step of creating the distraction by implementing manoeuvring means that cause a manoeuvre spacing the securing means apart,
a step of maintaining the distraction, by implementing the locking means.

28. Orthopaedic distraction method according to claim 26, wherein the patient's joint is a knee.

29. Orthopaedic distraction method according to claim 26, wherein the method comprises a step of generating an angulation between the securing means.

30. The orthopaedic distraction device according to claim 22,
wherein said at least two arms comprise:
a first arm, having a tubular wall that defines an elongated housing opening into an aperture, and
a second arm, fitted into said elongated housing through said aperture and guided in translation along said elongated housing,
wherein said at least one stop screw is chosen among:
an axial stop screw, to maintain the distraction, implanted parallel to the translational degree of freedom, in a bottom of the elongated housing located opposite the aperture, and
a side stop screw, to lock the translational clearance, implanted perpendicular to the translational degree of freedom, in the tubular wall of the first arm.
